(19) United States
(12) Patent Application Publication
(10) Pub. No.: US 2022/0401237 A1
(43) Pub. Date: Dec. 22, 2022

(54) Elastomeric Skin Contact Material that Contains and Releases CBD Oil

(71) Applicant: Gregory S. Bernhardt, Ivyland, PA (US)

(72) Inventor: Gregory S. Bernhardt, Ivyland, PA (US)

(21) Appl. No.: 17/832,648

(22) Filed: Jun. 5, 2022

Related U.S. Application Data
(60) Provisional application No. 63/208,941, filed on Jun. 9, 2021.

(51) Int. Cl. A61F 2/78; A61L 27/36; A61L 27/16; A61L 27/50; A61K 31/05

(52) U.S. Cl. CPC A61F 2/7812; A61L 27/3637; A61L 27/16; A61L 27/502; A61K 31/05; A61F 2002/785

(57) ABSTRACT

An improved composition for a thermoplastic elastomer that is used in a product that touches the skin. The thermoplastic elastomer contains a styrene block copolymer, a plasticizing oil, and CBD oil. The plasticizing oil and the CBD oil, in combination, comprise at least eighty percent of the thermoplastic elastomer by weight. The plasticizing oil and the CBD oil have the same, or similar, molecular weights. In that manner, the plasticizing oil and the CBD oil can form a homogenous mixture. The CBD oil saturates the thermoplastic elastomer and will slowly bloom out of the thermoplastic elastomer over time. The migration of the CBD oil brings the CBD oil to the exposed surfaces of the thermoplastic elastomer, therein enabling the CBD oil to be absorbed by the skin.

### RELATED APPLICATIONS

This application claims the benefit of U.S. Provisional Application No. 63/208,941, filed Jun. 9, 2021.

### BACKGROUND OF THE INVENTION

### 1. Field of the Invention

The present invention relates to elastomeric compounds, such as thermoplastic elastomers, that are used to cushion the interface between the skin and a secondary object. More particularly, the present invention relates to such elastomeric compound formulations where a desired substance is dissolved within the elastomeric compound, at a supersaturated concentration, causing the desired substance to slowly bloom to the surface of the elastomeric compound and be dispensed into the surrounding environment.

### 2. Description of the Prior Art

Bloom is a term of art in elastomers that refers to the migration of a liquid or solid material from the composition of the elastomer to the surface of the elastomer. Bloom is caused when an ingredient is added to an elastomeric compound formulation in a concentration that is in excess of its solubility. In such a situation, if the conditions for the supersaturated ingredient are more favorable in the presence of air than within the body of the elastomer, then the supersaturated ingredient will separate out of the elastomeric compound and migrate to the surface of the elastomer. The change in the saturation concentration gradient created at the air-exposed surface causes the supersaturated ingredient to migrate from within the elastomer compound toward the exterior surfaces. The bloom of the supersaturated ingredient will continue over time until the concentration of the blooming ingredient within the elastomeric compound falls to a concentration corresponding to the saturation point of the elastomeric compound. Bloom ceases when the concentration of the migrating substance in the elastomeric compound becomes equal to its solubility at a given temperature.

Thermoplastic elastomer is the generalized name given to a class of synthetic rubber. Thermoplastic elastomers can be formed into soft elastomeric gels by mixing plasticizing oils into the elastomeric polymer prior to cure. Some of the most common thermoplastic elastomers are made using styrene block copolymers that are mixed with paraffinic oils and/or white mineral oils. Styrene block copolymers are compatible with such petroleum-based oils. The compatibility of the styrene block copolymers and the petroleum-based oils results from the chemical nature of the midblock of the styrene block copolymers, which is very similar to the chemical nature of the paraffinic oil and/or mineral oil. The lower the molecular weight of the oil, the better the compatibility. By using a very low molecular weight oil, i.e., less than 400 g/mol in combination with high molecular weight styrene block copolymers, a useful formulation of a thermoplastic elastomer is created that can be readily pelletized and molded, yet remains very soft and pliant.

Thermoplastic elastomers are used to make interfaces between the skin and secondary objects. For example, thermoplastic elastomers are used in the making of limb liners for prosthetic limbs. In order for the prosthetic limb to function properly, the prosthetic limb must be firmly attached to the body. The most common method of attachment requires the use of a limb liner. The limb liner is pulled over the residual limb. The limb liner contains thermoplastic elastomers that conforms to the shape of the residual limb and creates a strong frictional attachment to the skin of the residual limb. As such, the limb liner acts as the anchor for retaining the limb prosthesis onto the residual limb.

One problem with using thermoplastic elastomers along a skin contact interface is that thermoplastic elastomers are waterproof and do not enable the skin to breathe. As such, the elastomeric material is often perforated and/or lined with some fabric that can wick away sweat and other moisture. This works only to a limited extent and lessens the grip that the thermoplastic elastomer creates against the skin. The moist environment between the skin and the thermoplastic elastomer can quickly begin to harbor bacteria. The moist environment created against the skin also softens the skin and makes the skin more prone to chafing. The result is that the product made from the thermoplastic elastomer must be cleaned and sanitized often. The cleaning often uses detergents that degrade the thermoplastic elastomer. Accordingly, the more times a product is laundered, the shorter its useful life becomes.

Cannabidiol oil, commonly known as CBD oil is a phytocannabinoid that is extracted from Cannabis plants. CBD oil has a molecular weight of approximately 314 g/mol, which is comparable to both light mineral oils and light paraffin oils. CBD oil, when topically applied to skin, has the useful effects of making the skin more pliable, deadening pain, and reducing inflammation.

The invention herein discloses a formulation and method of including CBD oil in the formation of thermoplastic elastomers and using the thermoplastic elastomers in the formation of products, such as limb liners, that are worn against the skin. In this manner, many of the health issues inherent in placing a thermoplastic elastomer against the skin can be reduced. The present invention is described and claimed below.

### SUMMARY OF THE INVENTION

The present invention is an improved composition for a thermoplastic elastomer that is used in a product that touches the skin, such as a limb liner for a prosthetic limb. The thermoplastic elastomer contains a styrene block copolymer, a first volume of a plasticizing oil, and a second volume of CBD oil. The plasticizing oil and the CBD oil, in combination, comprise at least eighty percent of the thermoplastic elastomer by weight. The plasticizing oil and the CBD oil have the same, or similar, molecular weights. In this manner, the plasticizing oil and the CBD oil can form a homogenous oil mixture during processing.

The CBD oil saturates the thermoplastic elastomer at molding temperatures. Accordingly, after molding, the CBD oil supersaturates the thermoplastic elastomer. As a result, the CBD oil will slowly bloom out of the thermoplastic elastomer over time. The migration of the CBD oil brings the CBD oil to the exposed surfaces of the thermoplastic elastomer, therein enabling the CBD oil to be absorbed by the skin being contacted. Once absorbed by the skin, the CBD oil helps to moisturize the skin, deaden pain, and reduce inflammation.

### BRIEF DESCRIPTION OF THE DRAWINGS

For a better understanding of the present invention, reference is made to the following description of an exemplary embodiment thereof, considered in conjunction with the accompanying drawings, in which:

FIG. 1 shows an exemplary embodiment of a limb liner shown in conjunction with a socket for a limb prosthesis;

FIG. 2 is an enlarged cross-sectional view of the limb liner as viewed in circle 2 of FIG. 1; and

FIG. 3 shows an exemplary overview for the methodology of making the thermoplastic elastomer used within the limb liner.

### DETAILED DESCRIPTION OF THE DRAWINGS

Although the present invention device and methodology can be used to make many products that touch the skin, such as gloves, socks, helmet pads and the like, in this description only one exemplary embodiment is illustrated. The exemplary embodiment is being shown for the purposes of explanation and description. The exemplary embodiment is selected in order to set forth one of the best modes contemplated for the invention. The illustrated embodiment, however, is merely exemplary and should not be considered as limiting when interpreting the scope of the appended claims.

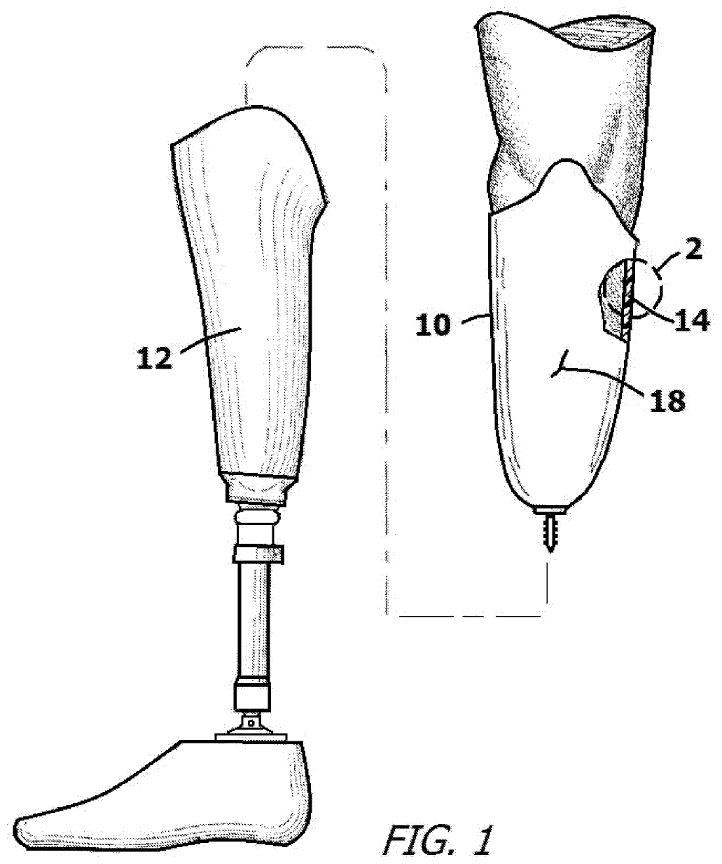

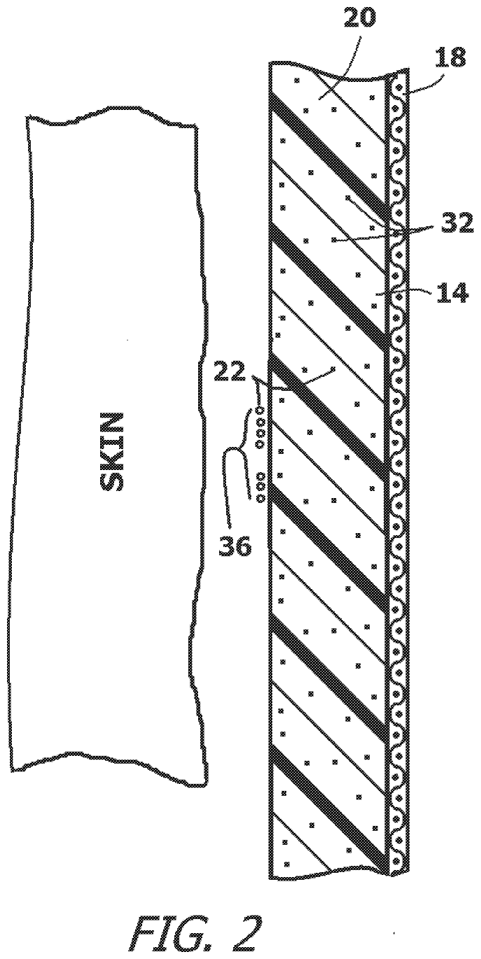

The present invention is a composition and methodology for making a thermoplastic elastomer that is used on a product that touches the skin. The thermoplastic elastomer can be used on hats, gloves, socks, sports pads, and the like. However, the present invention is particularly well suited for use on limb liners for prosthetic limbs. Accordingly, a limb liner is used as the exemplary embodiment in the illustrations. Referring to FIG. 1 and FIG. 2, a limb liner 10 is shown. When a person is fitted for a limb prosthesis 12, the limb liner 10 is typically placed over the portion of the residual limb that remains on the body. The limb liner 10 is pulled over the residual limb. The limb liner 10 contains a soft elastomeric layer 14 that is covered with a flexible substrate 18, such as an outer fabric liner. The flexible substrate 18 can be made from any pliable material including woven fabrics, non-woven fabrics, knitted and braided materials.

The soft elastomeric layer 14 is made from a thermoplastic elastomer 20 that contains, and slowly secretes, CBD oil 22. The CBD oil 22 is secreted, in part, through the process of plastic bloom. The phenomenon of bloom can be produced by adding CBD oil 22 to the formulation of the thermoplastic elastomer 20 in a concentration that supersaturates the thermoplastic elastomer 20 at skin temperature. External skin temperature is typically between 85 degrees Fahrenheit and 95 degrees Fahrenheit. Additionally, to promote the bloom of the CBD oil 22, the added CBD oil 22 has the inherent characteristic of precipitating out of solution more energetically in the presence of air than within the body of the thermoplastic elastomer 20. Accordingly, the CBD oil 22 contained within the thermoplastic elastomer 20 will slowly separate from thermoplastic elastomer 20 along exposed surfaces. The resulting change in the concentration gradient of the CBD oil 22, causes the CBD oil 22 to actively migrate toward the exterior surfaces of the soft elastomeric layer 14. The amount of CBD oil 22 that eventually migrates to the exposed surfaces of the soft elastomeric layer 14 is controlled by the amount of oversaturation, the volume of the thermoplastic elastomer 20, and the exposed surface area on the soft elastomeric layer 14. As such, the bloom of the CBD oil 22 from the thermoplastic elastomer 20 will continue until the concentration of the CBD oil 22 within the thermoplastic elastomer 20 falls to its saturation point. As the CBD oil 22 migrates to the exposed surfaces of the soft elastomeric layer 14, the CBD oil contacts the skin. The process of bloom is not instantaneous. Rather, depending upon such variables as the type of elastomer, surface area, volume, and degree of supersaturation, bloom may occur within a period of days to years before an equilibrium within the thermoplastic elastomer 20 is finally achieved.

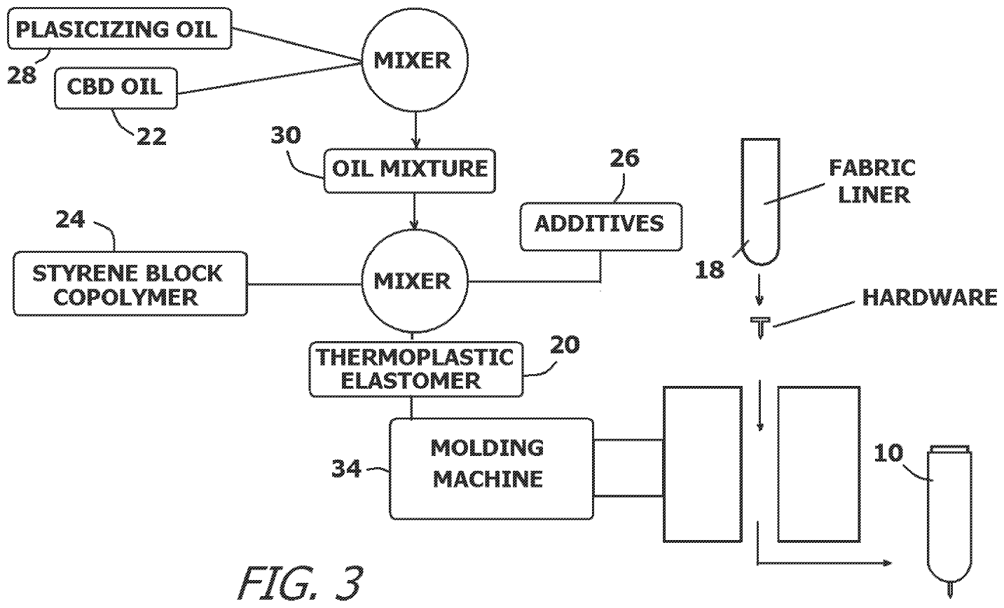

Referring to FIG. 3, in conjunction with FIG. 1 and FIG. 2, it can be seen that the thermoplastic elastomer 20 includes a styrene block copolymer 24 that is mixed with both a volume of plasticizing oil 28, a volume of CBD oil 22, and other additives 26. Non-limiting examples of a styrene block copolymer 24 include styrene-ethylene-butylene-styrene, styrene-ethylene-propylene-styrene, styrene-ethylene-ethylene/propylene-styrene, styrene-isobutylene-styrene, styrene-butadiene-styrene, styrene-isoprene-styrene, and combinations thereof. These examples of styrene block copolymers may or may not be maleated. The styrene block copolymer 24 has an average molecular weight in excess of 150,000 g/mol and preferably in excess of 200,000 g/mol. Preferred styrene block copolymers include styrene-ethylene-butylene-styrene (SEBS) and styrene-ethylene-ethylene/propylene-styrene (SEEPS) because these copolymers have olefinic mid-blocks that are capable of holding large amounts of a plasticizing oil 28.

The plasticizing oil 28 is selected to have a molecular weight generally equal to that of the CBD oil 22, that is, a molecular weight of between 310 g/mol and 320 g/mol. There are several paraffinic oils and mineral oils that embody such molecular weights. Peneteck LT mineral oil from Calumet Penreco of Dallas, Tex., and PD-23 WHITE OIL from Sonneborn, Inc. of Tarrytown, N.Y. are acceptable examples. In this manner, when the plasticizing oil 28 and the CBD oil 22 are mixed, a homogeneous mixture can be maintained where one oil does not float atop the other.

A volume of the plasticizing oil 28 is added to the styrene block copolymer 24. Within a range, the volume of plasticizing oil 28 that is added determines the durometer of the thermoplastic elastomer 20. In the formulation for the thermoplastic elastomer 20, the styrene block copolymer 24 is between 8% and 15% by weight. The CBD oil 22 is premixed with the plasticizing oil 28 to create an oil mixture 30. The plasticizing oil 28 and the CBD oils dissolve into one another only to a limited extent. Because the plasticizing oil 28 and the CBD oil 22 are selected to have the same general molecular weight, the oil mixture 30 remains homogenous during processing. The oil mixture 30 is between 80% and 90% of the overall formulation by weight. Within the oil mixture 30, nearly half of the volume can be CBD oil 22. As shown by Table 1 below, the volume of the CBD oil 22 can be as low as five percent and still have some degree of CBD oil bloom. The remaining percentages of the exemplary formulation can be one or more additives.

TABLE 1

| Ingredient | Percentage by Weight |
|---|---|
| Styrene block copolymer | 8%-13% |
| Plasticizing oil | 45%-80% |
| CBD oil | 45%-5% |
| Additives | 1%-3% |

Examples of additives 26 include adhesion promoters, biocides (antibacterials, fungicides, and mildewcides), anti-fogging agents, anti-static agents, bonding, blowing and foaming agents, dispersants, fillers and extenders, impact modifiers, initiators, lubricants, micas, pigments, colorants, and dyes, release agents, silanes, titanates, and zirconates, slip and anti-blocking agents, stabilizers; stearates, ultraviolet light absorbers, viscosity regulators, waxes, and combinations thereof.

The styrene block copolymer 24 is a petroleum derived material. Although the plasticizing oil 28 and the CBD oil 22 have the same general molecular weight, the CBD oil 22 is an organic plant-based oil and the plasticizing oil 28 is a synthetic petroleum-based oil. Accordingly, the CBD oil 22 is absorbed less readily by the styrene block copolymer 24 than is the petroleum-based plasticizing oil 28. Accordingly, not all the CBD oil 22 is absorbed by the styrene block copolymer 24 and the CBD oil 22 saturates the styrene block copolymer 24 when the styrene block copolymer is molten. Accordingly, as the thermoplastic elastomer 20 cures and solidifies, the CBD oil 22 is present in a concentration that supersaturates the thermoplastic elastomer 20. Furthermore, there are undissolved microdroplets 32 of CBD oil 22 trapped within the cured thermoplastic elastomer 20.

The thermoplastic elastomer 20 is introduced into a molding machine 34. The molding machine 34 molds the thermoplastic elastomer 20 onto one or more fabric liners, therein creating the soft elastomeric layer 14. As the thermoplastic elastomer 20 cures in the molding machine 34, the thermoplastic elastomer 20 bonds to the outer flexible substrate 18, therein completing the limb liner 10.

Referring back to FIG. 2, it can be seen that once the thermoplastic elastomer 20 is molded onto a limb liner 10, surfaces of the thermoplastic elastomer 20 are exposed to air. Due to the supersaturation of the CBD oil 22 in the thermoplastic elastomer 20, the CBD oil 22 will slowly permeate out of the soft elastomeric layer 14 along surfaces that are exposed to air. Additionally, microdroplets 32 of undissolved CBD oil 22 within the thermoplastic elastomer 20 are slowly driven to the exterior surfaces of the soft elastomeric layer 14 as the soft elastomeric layer 14 is repeatedly stretched and compressed while being worn. The CBD oil 22, therefore, slowly forms free microdroplets 36 of CBD oil 22 on the exposed surfaces of the soft elastomeric layer 14. These free microdroplets 36 are absorbed by the skin through direct physical contact.

Once the CBD oil 22 reaches the skin, the CBD oil 22 is absorbed by the skin and underlying tissue. The CBD oil 22 then acts to moisturize the skin, block pain receptors, and help reduce swelling. The CBD oil 22 exits the thermoplastic elastomer 20 very slowly. The period for release can last years, which is longer than the average life span of a limb liner 10. Furthermore, since the CBD oil 22 is both dissolved in the thermoplastic elastomer 20 and entrapped within the thermoplastic elastomer 20, the CBD oil 22 cannot be washed out of the limb liner 10. Rather, upon laundering, exposed CBD oil 22 is removed, but newer CBD oil 22 migrates to the exposed surfaces.

It will be understood that the embodiment of the present invention that is illustrated and described is merely exemplary and that a person skilled in the art can make many variations to that embodiment. The present invention can be used on any product that contains padding made from a thermoplastic elastomer, wherein the padding touches the skin. Such products, include, but are not limited to sports padding, padded gloves, padded socks, hat brims, and the like. All such embodiments are intended to be included within the scope of the present invention as defined by the claims.

What is claimed is:

1. A padded liner for a prosthetic limb, comprising:
at least one layer of a thermoplastic elastomer that contains a styrene block copolymer, a first volume of a plasticizing oil, and a second volume of CBD oil, wherein said CBD oil blooms from said thermoplastic elastomer over a prolonged period of time.

2. The padded liner according to claim 1, wherein said plasticizing oil and said CBD oil in combination comprise at least eighty percent by weight of the thermoplastic elastomer by weight.

3. The padded liner according to claim 1, wherein said CBD oil comprises at least five percent of said thermoplastic polymer by weight.

4. The padded liner according to claim 1, wherein both said plasticizing oil and said CBD oil have a molecular weight of between 310 g/mol and 320 g/mole.

5. The padded liner according to claim 1, further including at least one flexible substrate bonded to said thermoplastic elastomer.

6. The padded liner according to claim 1, wherein said styrene block copolymer is selected from a group consisting of styrene-ethylene-butylene-styrene, styrene-ethylene-propylene-styrene, styrene-ethylene-ethylene/propylene-styrene, styrene-isobutylene-styrene, styrene-butadiene-styrene, styrene-isoprene-styrene, and combinations thereof.

7. The padded liner according to claim 6, wherein said styrene block copolymer has an average molecular weight in excess of 150,000 g/mol.

8. The padded liner according to claim 1, wherein said plasticizing oil is selected from a group consisting of paraffinic oils and mineral oils.

9. The padded liner according to claim 1, wherein said CBD oil supersaturates said thermoplastic elastomer.

10. A padded product that is worn in contact with the skin, comprising:
at least one soft elastomeric layer, wherein said soft elastomeric layer is a thermoplastic elastomer that contains a styrene block copolymer, plasticizing oil, and CBD oil;
a flexible substrate that covers at least part of said soft elastomer layer, where said thermoplastic elastomer is bonded to said flexible substrate.

11. The padded product according to claim 10, wherein said CBD oil migrates out of said soft elastomeric layer over a prolonged period of time.

12. The limb liner according to claim 11, wherein said CBD oil supersaturates said thermoplastic elastomer.

13. The limb liner according to claim 10, wherein said plasticizing oil and said CBD oil in combination comprise at least eighty percent by weight of the thermoplastic elastomer by weight.

14. The limb liner according to claim 10, wherein said CBD oil comprises between five percent and forty five percent of said thermoplastic elastomer by weight.

15. The limb liner according to claim 10, wherein both said plasticizing oil and said CBD oil have a molecular weight of between 310 g/mol and 320 g/mole.

16. The limb liner according to claim 10, wherein said styrene block copolymer is selected from a group consisting of styrene-ethylene-butylene-styrene, styrene-ethylene-propylene-styrene, styrene-ethylene-ethylene/propylene-styrene, styrene-isobutylene-styrene, styrene-butadiene-styrene, styrene-isoprene-styrene, and combinations thereof.

17. The limb liner according to claim 16, wherein said styrene block copolymer has an average molecular weight in excess of 150,000 g/mol.

18. The limb liner according to claim 10, wherein said plasticizing oil is selected from a group consisting of paraffinic oils and mineral oils.
